(19) United States
(12) Patent Application Publication
(10) Pub. No.: US 2026/0328664 A1
(43) Pub. Date: Oct. 22, 2026

(54) METHOD, DEVICE, ELECTRONIC DEVICE, AND STORAGE MEDIUM FOR PROCESSING STRESS DATA OF TEST-PIECE BASED ON THE MEAN STRESS DISTRIBUTION

(71) Applicant: Hunan University of Science and Technology, Xiangtan (CN)

(72) Inventors: Yongping Jin, Qiandongnan Prefecture (CN); Gan Wang, Xiangtan (CN); Deshun Liu, Xiangtan (CN)

(21) Appl. No.: 19/686,976

(22) Filed: May 25, 2026

(30) Foreign Application Priority Data
Jan. 26, 2026 (CN) 202610098760.3

(51) Int. Cl. G01N 3/02; G01N 3/30

(52) U.S. Cl. CPC G01N 3/02; G01N 3/30; G01N 2203/0075

(57) ABSTRACT

Provided are a stress data processing method and related equipment for test-piece based on the mean stress distribution. The stress data processing method includes: determining a mean stress distribution of a target test-piece; constructing a dynamic response reconstruction model based on the mean stress distribution; constructing a stress distribution function of the target test-piece through a preset optimization algorithm; substituting the stress distribution function into the dynamic response reconstruction model for calculation, and determining a target stress data of the target test-piece; and testing and judging applicability or failure risk of the target test-piece under preset working conditions based on the target stress data. Through the above method steps, it is possible to analyze the true stress state of the test-piece under dynamic loading conditions, provide data basis for the performance evaluation of the test-piece, and improve the accuracy and efficiency of the stress analysis of the test-piece.

### CROSS-REFERENCE TO RELATED APPLICATIONS

This application claims priority to Chinese Patent Application No. 202610098760.3, filed on Jan. 26, 2026, which is hereby incorporated by reference in its entirety.

### TECHNICAL FIELD

The present disclosure relates to the field of dynamic mechanical performance testing of solid materials, and in particular, to a method, device, electronic device, and storage medium for processing stress data of test-piece based on mean stress distribution.

### BACKGROUND

Split Hopkinson Pressure Bar (SHPB) is a commonly used technique for testing the dynamic mechanical properties of materials in the strain rate range of 10²-10⁴ s⁻¹. The basic principle of Split Hopkinson Pressure Bar test is based on one-dimensional elastic wave dynamics theory. The incident wave, reflected waves, and transmitted wave data obtained through strain measurement on the SHPB elastic rod are usually calculated using existing data processing methods such as two wave and three wave to reconstruct the stress, strain, and strain rate of the test-piece. Based on the reconstructed stress-strain curve of the test-piece, the mechanical properties such as yield strength, compressive strength, and elastic modulus of the test-piece material are determined.

The traditional SHPB test method generally calculates the stress signal at a single point at the end of the test-piece and assumes that the internal stress of the test-piece is uniformly distributed to complete the analysis and derivation of stress, strain, and strain rate. This type of testing approach is difficult to truly reflect the internal stress state of the test-piece in high strain rate impact environments, especially in cases where stress propagation has temporal evolution and spatial distribution differences during the impact process. Relying solely on end measurement point data for overall stress estimation often results in error bias. In addition, existing methods often do not construct corresponding force function models based on real internal force distribution characteristics, and lack the process of establishing dynamic response reconstruction models through unified physical meaning, resulting in differences between the performance evaluation results obtained in experiments and the dynamic mechanical properties of test-piece, rendering it difficult to accurately identify the applicability or potential failure risks of test-piece under different working conditions.

Therefore, the stress-strain curves of test-piece obtained through existing stress data processing methods based on the average stress values of two-end or single-end face stress have problems of high reconstruction errors and low data accuracy.

### SUMMARY

The present disclosure provides a method for processing stress data of test-piece based on mean stress distribution so as to solve the problems of high reconstruction error and low data accuracy in the stress-strain curve of test-piece obtained through existing stress data processing methods based on the average stress values of two-end faces or single-end face stress.

Compared with existing technologies, the beneficial effect of the present disclosure is that it can avoid the systematic errors introduced by traditional stress data processing methods based on the average stress values of two-end faces or single-end face stress when the equivalent conditions of the end face are insufficient, and improve the accuracy and stability of the overall stress state reconstruction of the test-piece, so that dynamic indicators such as stress, strain, and strain rate have consistent calculation basis, and improve the accuracy of the stress-strain curve of the test-piece.

In a first aspect, the present disclosure provides a stress data processing method for test-piece based on the mean stress distribution, including:
determining a mean stress distribution of a target test-piece;
constructing a dynamic response reconstruction model based on the mean stress distribution;
constructing a stress distribution function of the target test-piece through a preset optimization algorithm;
substituting the stress distribution function into the dynamic response reconstruction model for calculation, and determining a target stress data of the target test-piece; and
testing and judging applicability or failure risk of the target test-piece under preset working conditions based on the target stress data.

In some embodiments of the present disclosure, determining a mean stress distribution of a target test-piece includes:
real time collecting a force response of the target test-piece at a preset position, obtaining a dynamic response data of the target test-piece, and the dynamic response data is configured to describe an end force change of the target test-piece; and
calculating and processing an internal stress distribution of the target test-piece based on the dynamic response data to obtain the mean stress distribution.

In some embodiments of the present disclosure, the dynamic response reconstruction model includes stress reconstruction model, a strain reconstruction model, and a strain rate reconstruction model; where constructing a dynamic response reconstruction model based on the mean stress distribution includes:
determining target correlation parameters of the target test-piece based on the mean stress distribution, and the target correlation parameters are configured to uniformly characterize a dynamic stress state of the target test-piece; and
constructing the stress reconstruction model, the strain reconstruction model, and the strain rate reconstruction model respectively based on the target correlation parameters.

In some embodiments of the present disclosure, constructing a stress distribution function of the target test-piece through a preset optimization algorithm includes:
determining an objective function based on the dynamic response data and stress distribution mean of the target test-piece, and the objective function is configured to characterize the internal stress distribution characteristics of the target test-piece; and
solving and calculating the objective function using the preset optimization algorithm to obtain the stress distribution function.

In some embodiments of the present disclosure, substituting the stress distribution function into the dynamic response reconstruction model for calculation and determining a target stress data of the target test-piece includes:
inputting the stress distribution function as an input parameter into the dynamic response reconstruction model, calculating the stress response of the target test-piece during dynamic loading, and obtaining a force response calculation data;
extracting and processing characteristics of an internal force variation of the target test-piece over time and characteristics along a spatial position based on the force response calculation data, and obtaining time distribution feature data and spatial distribution feature data; and
analyzing an overall stress state of the target test-piece based on the time distribution feature data and the spatial distribution feature data and obtaining the target stress data.

In some embodiments of the present disclosure, analyzing an overall stress state of the target test-piece and obtaining the target stress data based on the time distribution feature data and the spatial distribution feature data includes:
determining a first force feature data based on the time distribution feature data, and the first force feature data is configured to reflect the dynamic stress variation law of the target test-piece;
determining a second force feature data based on the spatial distribution feature data, and the second force feature data is configured to reflect an internal force distribution state of the target test-piece; and
obtaining the target stress data based on a correlation calculation between the first force feature data and the second force feature data, and characterizing the overall stress state of the target test-piece using the target stress data.

In some embodiments of the present disclosure, testing and judging applicability or failure risk of the target test-piece under preset working conditions based on the target stress data includes:
constructing a dynamic stress-strain response curve of the target test-piece based on the target stress data;
determining a mechanical performance data of the target test-piece under preset working conditions based on the dynamic stress-strain response curve, and the mechanical performance data includes stress threshold, structural limit, and deformation characteristics; and
testing and judging the applicability or failure risk of the target test-piece under preset working conditions based on the stress threshold, structural limit, and deformation characteristics of the target test-piece under preset working conditions.

In a second aspect, the present disclosure further provides a stress data processing device for test-piece based on mean stress distribution, where the stress data processing device for test-piece based on mean stress distribution includes:
a first acquisition module, configured to determine a mean stress distribution of a target test-piece;
a first construction module, configured to construct a dynamic response reconstruction model based on the mean stress distribution;
a second construction module, configured to construct a stress distribution function of the target test-piece through a preset optimization algorithm;
a first determination module, configured to substitute the stress distribution function into the dynamic response reconstruction model for calculation, and determine a target stress data of the target test-piece; and
a testing module, configured to test and judge applicability or failure risk of the target test-piece under preset working conditions based on the target stress data.

In a third aspect, the present disclosure provides an electronic device, which includes: a memory, a processor, and a computer program that is stored on the memory and executable on the processor, when the processor executes the computer program, it implements steps of the stress data processing method for test-piece based on the mean stress distribution provided by the present disclosure.

In a fourth aspect, the present disclosure provides a computer-readable storage medium, on which a computer program is stored; when the computer program is executed by a processor, it implements steps of the stress data processing method for test-piece based on the mean stress distribution provided by the present disclosure.

The present disclosure determines the mean stress distribution of the target test-piece; constructs a dynamic response reconstruction model based on the mean stress distribution; constructs the stress distribution function of the target test-piece through the preset optimization algorithm; substitutes the stress distribution function into the dynamic response reconstruction model for calculation, and determine the target stress data of the target test-piece; based on the target stress data, tests and judges the applicability or failure risk of the target test-piece under preset working conditions.

Compared with existing technologies, the beneficial effect of the present disclosure is that it can avoid the systematic errors introduced by traditional stress data processing methods based on the average stress values of two-end faces or single-end face stress when the equivalent conditions of the end face are insufficient, and improve the accuracy and stability of the overall stress state reconstruction of the test-piece, so that dynamic indicators such as stress, strain, and strain rate have consistent calculation basis, and improve the accuracy of the stress-strain curve of the test-piece.

### BRIEF DESCRIPTION OF DRAWINGS

In order to provide a clearer explanation of the embodiments of the present disclosure or the technical solutions in the prior art, a brief introduction will be given to the accompanying drawings required for the description of the embodiments or the prior art. It is obvious that the accompanying drawings described below are only some embodiments of the present disclosure. For those skilled in the art, other drawings can be obtained based on these drawings without creative work.

FIG. 1 is a flowchart of a stress data processing method for test-piece based on the mean stress distribution provided by the present disclosure.

FIG. 2 is a schematic diagram of a SHPB experimental device provided by the present disclosure.

FIG. 3 is a structural schematic diagram of a stress data processing device for test-piece based on mean stress distribution provided by the present disclosure.

FIG. 4 is a structural schematic diagram of an electronic device provided by the present disclosure.

### DESCRIPTION OF EMBODIMENTS

Below, the technical solutions in the embodiments of the present disclosure will be clearly and completely described in combination with the accompanying drawings. Obviously, the described embodiments are only a part of the embodiments of the present disclosure, not all of them. Based on the embodiments of the present disclosure, all other embodiments obtained by those skilled in the art without creative work are within the protection scope of the present disclosure.

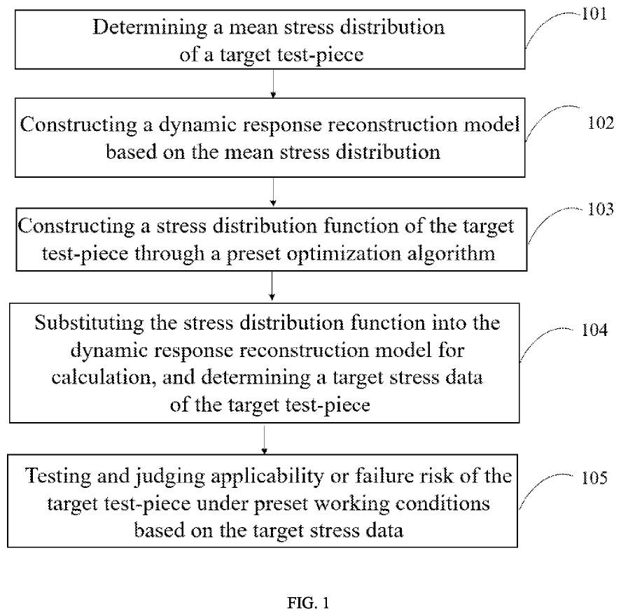

As shown in FIG. 1, it is a flowchart of a stress data processing method for test-piece based on mean stress distribution provided by the present disclosure. The stress data processing method for test-piece based on mean stress distribution includes the following steps.

### 101. Determining a Mean Stress Distribution of a Target Test-Piece.

In the embodiments of the present disclosure, the stress data processing method for test-piece based on mean stress distribution can be applied to a stress data processing platform based on the mean stress distribution. The stress data processing platform based on the mean stress distribution has functions such as stress data processing, stress data transmission and reception, and stress data memory and storage. It can be constructed based on servers or server clusters, which can be electronic devices with stress data processing capabilities.

In this embodiment, the target test-piece stress testing platform based on the mean stress distribution can detect the end stress wave signals of the target test-piece during SHPB impact loading in real time through strain gauges, fiber optic sensors, or dynamic response collectors installed at the loading and support ends, and convert the detected electrical signals into processable dynamic response data. For example, it is essentially the collection of raw information on the stress state of the target test-piece, which serves as the fundamental data source for subsequent calculations of internal stress distribution and model construction.

The target test-piece can refer to a tested sample whose impact response and mechanical properties are verified through SHPB dynamic testing. It can be metal materials, rock materials, composite materials, engineering structural components, or other industrial materials with mechanical testing requirements. During the dynamic impact loading process, stress propagation, stress wave superposition, and plastic yield characteristics will occur inside the target test-piece, all of which can be identified and analyzed through the target test-piece stress testing platform based on the mean stress distribution mentioned above.

The mean stress distribution mentioned above can be used to characterize the true internal stress state of the target test-piece under dynamic impact. In an implementation mode, the target test-piece stress testing platform based on the mean stress distribution can estimate the stress at different spatial positions inside the test-piece through inversion analysis and internal stress distribution calculation based on the collected dynamic response data at the end of the test-piece, and finally obtain the statistical equilibrium of the internal stress values in the entire domain.

It can be understood that the mean stress distribution mentioned above can enable stress, strain, and strain rate analysis to be conducted in the same physical sense in this embodiment, thereby avoiding the problem of overall stress inversion deviation caused by traditional testing methods relying only on the end data.

### 102. Constructing a Dynamic Response Reconstruction Model Based on the Mean Stress Distribution.

In the embodiments of the present disclosure, a mathematical model or a descriptive function with engineering calculation significance can be established through the mean stress distribution. In an implementation mode, the target test-piece stress testing platform based on the mean stress distribution is reshaped through basic parameter combinations, variable associations, and response relationships to form a model structure for further calculations. For example, after obtaining the mean stress distribution, the target test-piece stress testing platform based on the mean stress distribution will convert the stress characteristics of the target test-piece into a set of input parameters that can be dynamically solved, and this is used to construct the dynamic response reconstruction model for subsequent stress field solving.

The above dynamic response reconstruction model can refer to a unified model structure that can output stress, strain and strain rate at the same time, including but not limited to a stress reconstruction model, a strain reconstruction model and a strain rate reconstruction model. Where the above dynamic response reconstruction model does not rely on the traditional three wave method based on averaging stress at two-end face of the test-piece, nor does it rely on the two-wave method based on equivalent treatment method of single-end stress and assuming equal stress at two ends of the test-piece. Instead, it establishes conditions of unified physical meaning through the distribution mean value, so that the three types of dynamic indicators have consistent calculation basis.

### 103. Constructing a Stress Distribution Function of the Target Test-Piece Through a Preset Optimization Algorithm.

In the embodiments of the present disclosure, the preset optimization algorithm can be used to fit a calculation strategy of internal force distribution characteristics of the target test-piece, aiming to satisfy the continuity of the internal force distribution function and the real mechanical response law of the test-piece by solving a objective function, including but not limited to iterative optimization, variational fitting, minimum error convergence criterion or other calculation strategies that can achieve distribution fitting.

The above stress distribution function can be a function expression or a characteristic relationship obtained by solving the objective function through the preset optimization algorithm to describe the spatial distribution law of the internal stress of the test-piece, and the stress distribution function is used to reflect the stress levels, stress gradient changes, and peak stress positions corresponding to different spatial positions of the target test-piece during dynamic loading. It can be used as an input parameter in the subsequent dynamic response reconstruction model. For example, when one end of the target test-piece is subjected to a shock wave, the internal stress will exhibit waveform changes along the propagation direction, which can be described by the stress distribution function, thereby providing a basis for subsequent overall stress calculation.

104. Substituting the Stress Distribution Function into the Dynamic Response Reconstruction Model for Calculation, and Determining a Target Stress Data of the Target Test-Piece.

In the embodiments of the present disclosure, the target test-piece stress testing platform based on the mean stress distribution can input the stress distribution function into the dynamic response reconstruction model. Based on the time history changes of the impact loading process, the internal stress response of the test-piece can be solved. In an implementation mode, the solving process can include information such as changes in stress over time, stress propagation speed, waveform superposition, and the synergistic effect of internal model parameters, which are used to generate global response data of the dynamic stress state of the test-piece.

### 105. Testing and Judging Applicability or Failure Risk of the Target Test-Piece Under Preset Working Conditions Based on the Target Stress Data.

In the embodiments of the present disclosure, the above-mentioned preset operating conditions may refer to the pre-set working environment conditions of the test-piece, including but not limited to impact strength, loading rate, strain rate level, temperature range, or other testing scenario parameters. It can be understood that the above preset operating conditions can be used to determine the stress analysis boundary of the target test-piece, so that the evaluation behavior of the target stress data has a clear test object and target.

In an implementation mode, the target test-piece stress testing platform based on mean stress distribution can be used to determine whether the target test-piece meets the stress bearing requirements under preset working conditions or whether there is a risk of entering a failure boundary based on the target stress data, stress-strain response curve characteristic points, and dynamic performance indicators.

In the embodiments of the present disclosure, the mean stress distribution of the target test-piece is determined; THE dynamic response reconstruction model is constructed based on the mean stress distribution; the stress distribution function of the target test-piece is constructed through the preset optimization algorithm; the stress distribution function is substituted into the dynamic response reconstruction model for calculation, and the target stress data of the target test-piece is determined; the applicability or failure risk of the target test-piece under preset working conditions is tested and judged based on the target stress data. Through the above method steps, it is possible to analyze the true stress state of the test-piece under dynamic loading conditions, thereby providing data basis for the performance evaluation of the test-piece, and improving the accuracy and efficiency of the stress analysis of the test-piece.

In some embodiments of the present disclosure, in step of determining the mean stress distribution of the target test-piece, real-time collection of a force response of the target test-piece at a preset position can also be performed to obtain a dynamic response data of the target test-piece, and the dynamic response data is used to describe an end stress change of the target test-piece; based on the dynamic response data, an internal stress distribution of the target test-piece is calculated and processed to obtain the mean stress distribution.

In the embodiments of the present disclosure, the preset position may refer to a data acquisition point on a force transmission path corresponding to the end of the target test-piece. This position is usually selected in an end face area where the test-piece is in contact with an incident bar or a transmission bar, and is used to collect the force signal of the test-piece during the dynamic impact loading process.

In an implementation mode, the preset position can be based on the stress wave propagation law of the target test-piece, where the shock wave first enters from the end of the test-piece and propagates inward. Therefore, the end position can directly reflect the stress changes of the test-piece at the moment of impact. By setting the preset position, it can be ensured that the collected data has temporal integrity and representative impact response.

For example, in SHPB testing, the stress waveform at the end face of the target test-piece is obtained by collecting data at the preset position.

The above force response can refer to characteristics of the stress wave signal generated by the target test-piece under dynamic loading, including but not limited to the peak force, rising edge shape, waveform change trend, and duration of force, which are used to reflect the actual stress state of the test-piece under impact loading. It can be understood that the above force response is usually reflected in the form of resistance strain signals, fiber optic photosensitive changes, or stress wave signals generated at the end face of the test-piece, which can reflect the propagation characteristics and force intensity changes of shock waves after they enter the test-piece. In an implementation mode, the target test-piece stress testing platform based on the mean stress distribution can continuously record the stress response signal at the end of the test-piece at a high sampling frequency during the impact loading testing process, thus ensuring that the collected data covers the complete process of shock wave generation, propagation, peak appearance, energy attenuation, etc.

The dynamic response data collected through the above method can be used to describe the temporal signal data of the stress changes at the end of the test-piece. It should be noted that the dynamic response data can be a continuous response curve that changes over time, reflecting the changes in the bearing capacity and internal stress propagation law of the test-piece under impact loading, including but not limited to the characteristics of stress peak, response duration, waveform structure, rising edge shape, etc. It is the data source for solving the internal stress distribution of the test-piece.

The internal force distribution can refer to the force change state generated along different spatial positions inside the target test-piece under dynamic impact loading, that is, the shock wave propagates inward from the end of the test-piece. During the process, due to material properties, geometric structure, and energy attenuation, there will be differences in the peak force and change process at various locations inside the test-piece. By analyzing the dynamic response data and calculating the internal stress propagation characteristics, the stress magnitude and change trend corresponding to different locations inside the test-piece can be obtained, thus forming the internal stress distribution.

By the above method steps, it is possible to avoid the inversion bias caused by the traditional assumption of equivalent end faces and improve the accuracy of calculating the true bearing capacity of the test-piece under dynamic loading conditions.

In an implementation mode, in step of constructing a dynamic response reconstruction model based on the mean stress distribution, it further includes determining target correlation parameters of the target test-piece based on the mean stress distribution, and the target correlation parameters are used to uniformly characterize a dynamic stress state of the target test-piece; based on the target correlation parameters, the stress reconstruction model, the strain reconstruction model, and the strain rate reconstruction model are constructed respectively.

In the embodiments of the present disclosure, the above-mentioned target correlation parameters may refer to characteristic parameters determined based on the mean stress distribution of the target test-piece for uniformly describing the force variation law of the test-piece under dynamic loading conditions. They can be comprehensively quantified based on the temporal and spatial variation characteristics of the internal stress state of the target test-piece, including but not limited to stress characteristic values reflecting the stress level of the test-piece, associated variables used to describe the trend of deformation rate changes, and other data.

The above dynamic stress state can refer to the true stress variation behavior of the target test-piece during the impact loading process, including the growth process of stress over time, peak occurrence time, internal stress wave propagation trend, and material strain response characteristics.

In an implementation mode, after obtaining the target correlation parameters, the target test-piece stress testing platform based on the mean stress distribution uses these parameters as a unified input basis to establish a model for solving the stress, strain, and strain rate response relationship of the test-piece during dynamic loading.

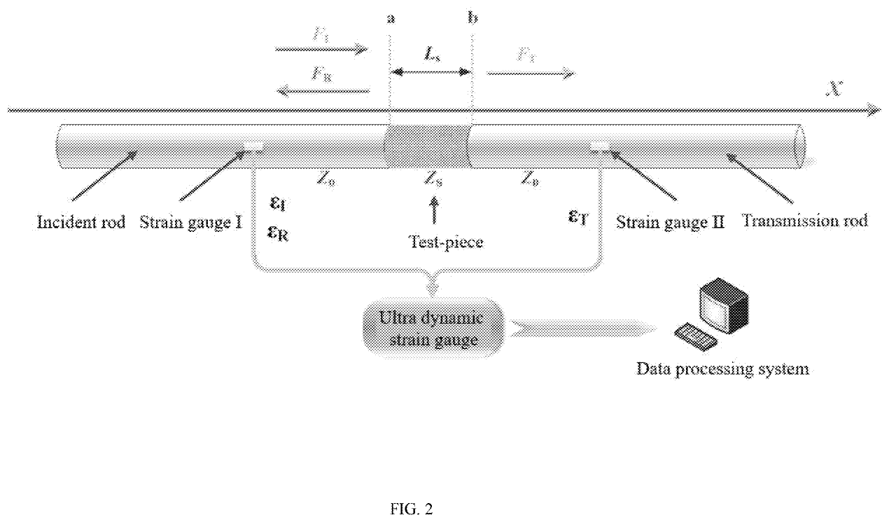

In an implementation mode, the stress reconstruction model, strain reconstruction model, and strain rate reconstruction model can be obtained through the following formulas:

$$\begin{matrix}{σ}_{S} ( t ) = \frac{1}{{L}_{S}} {\int }_{0}^{{L}_{S}} {σ}_{S} ( t , x ) dx \\ {ε}_{S} ( t ) = \frac{1}{{L}_{S}} {\int }_{0}^{{L}_{S}} {ε}_{S} ( t , x ) dx = \frac{1}{{L}_{S} {Z}_{0}} {\int }_{0}^{t} ( {F}_{I} ( t ) - {F}_{R} ( t ) - {F}_{T} ( t ) ) dt \\ {\dot{ε}}_{S} ( t ) = \frac{1}{{L}_{S} {Z}_{0}} ( {F}_{I} ( t ) - {F}_{R} ( t ) - {F}_{T} ( t ) )\end{matrix} \}$$

where, σ_{S}(t) is the stress reconstruction model, ε_{S}(t) is the strain reconstruction model, and \dot{ε}_{S}(t) is the strain rate reconstruction model, which can be explained according to the schematic diagram of the SHPB experimental device shown in FIG. 2. In the equation, σ_{S}(t,x) and ε_{S}(t,x) are the stress data and strain data of the target test-piece at time t and and position x, that is, the stress distribution function and strain distribution function of the target test-piece are at time t; L_{S} is a length of the target test-piece; a cross-sectional position of the target test-piece is measured form a left end a to a right end b, 0≤x≤L_{S}; Z₀ is a wave resistance of the incident bar and transmission bar in SHPB device; F_{I}(t), F_{R} (t) and F_{T} (t) are, the incident waves, reflected waves, and transmitted waves extracted from the incident bar and the transmission bar in the experiment, respectively.

It should be noted that before testing, the elastic modulus, cross-sectional areas, wave resistances Z₀ of the input bar and output bar, and test-piece length and test-piece cross-sectional area can be measured in advance according to existing technology. The test-piece length and cross-sectional area are L_{S} and A_{S} respectively. Therefore, in this experiment, the incident wave generated by the impact of the hammer on the input bar can propagate from left to right inside the input bar. During this process, two strain signals can be obtained by attaching two strain gauges to the input and output bars, respectively. Then, with the help of reasonable incident wave wavelength and input bar and output bar length design, the time b that the incident wave, reflected wave, transmitted wave, and stress wave pass through the test-piece can be extracted from the two strain signals, and the stress data of the test-piece can be calculated based on the extracted data.

In an implementation mode, the above stress reconstruction model can be used to solve the distribution relationship of internal stress of the test-piece with time and space changes during dynamic loading process based on target correlation parameters and stress distribution mean. Through the above stress reconstruction model, the true bearing pressure inside the test-piece can be restored, no longer limited to single point values at the end measurement points, and the overall stress level of the test-piece can be presented in a dynamic form.

The above strain reconstruction model can be used to determine the deformation characteristics of the test-piece during impact loading based on target correlation parameters, including compression, tension, or local plastic deformation state of the test-piece material after being subjected to shock waves, reflecting the stiffness characteristics and impact resistance of the test-piece.

The above strain rate reconstruction model can be used to determine the deformation rate variation law of the test-piece during the impact process based on the target correlation parameters. Through the above strain rate reconstruction model, the impact resistance performance of the test-piece in high strain rate environments can be evaluated.

Through the above method steps, it is possible to reconstruct the three dynamic indicators of stress, strain, and strain rate simultaneously on the basis of unified correlation parameters, thereby avoiding the accumulation of bias in traditional methods, improving the accuracy of test-piece dynamic behavior evaluation, and providing reliable basis for test-piece impact strength analysis, performance limit identification, and engineering applicability judgment.

In an implementation mode, in step of constructing a stress distribution function of the target test-piece through a preset optimization algorithm, it further includes determining an objective function based on the dynamic response data and stress distribution mean of the target test-piece. The objective function is used to characterize the internal stress distribution characteristics of the target test-piece; by using the preset optimization algorithm, the objective function is solved and calculated to obtain the stress distribution function.

In the embodiments of the present disclosure, the above objective function may be a calculation expression or a characteristic relationship constructed based on the dynamic response data of the test-piece and the mean stress distribution to characterize the internal stress distribution law of the target test-piece. Based on the dynamic response data collected in real-time at the end of the test-piece; by introducing the mean internal stress distribution information, the function can fit and correlate the stress areas inside the test-piece that are not directly collected.

In an implementation mode, when the test-piece is subjected to shock wave input, the waveform changes of the end signal contain information about the internal stress propagation process. The objective function transforms the characteristics of the internal stress gradient, peak change position, and stress concentration section into solvable data correlations through this information.

The above-mentioned internal force distribution characteristics can refer to the force variation state and force distribution law of the target test-piece at different spatial positions during the impact loading process, including but not limited to the peak force size at each position, the attenuation or enhancement trend of internal force, the concentrated area of force, delayed behavior of wave propagation, and differences in internal mechanical response. These characteristics can be derived and identified by combining dynamic response data with the overall force law of the test-piece.

In an implementation mode, the target test-piece stress testing platform based on the mean stress distribution is iteratively fitted and optimized using the preset optimization algorithm to determine the stress distribution function that can truly characterize the internal stress distribution relationship of the test-piece.

In an implementation mode, the above preset optimization algorithm can be obtained by setting the stress distribution function in the target test-piece to always maintain a minimum difference between the stress values at two ends and the test stress values at contact ends of the input bar, output bar, and test-piece. y is obtained by optimizing the following equation:

$${{min}^{{e}_{ab}}}_{y ∈ ( - 1 , 1 )} ( t ) = {min}_{y ∈ ( - 1 , 1 )} ( \begin{matrix}\vert {σ}_{s} ( t , x = 0 ) - \frac{1}{{A}_{S}} ( {F}_{I} ( t ) + {F}_{R} ( t ) ) \vert + \\ \vert {σ}_{s} ( t , x = {L}_{S} ) - \frac{1}{{A}_{S}} ( {F}_{T} ( t ) \vert\end{matrix} ) = {min}_{y ∈ ( - 1 , 1 )} \{ \begin{matrix}\begin{matrix}\vert \frac{1}{{A}_{S}} [ ( 1 + y ) {F}_{I} ( t ) + {\sum }_{i = 1}^{n} ( 1 - {y}^{2} ) \\ {( - y )}^{2 t - 1} {F}_{I} ( t - 2 ib ) ] - \frac{1}{{A}_{S}} ( {F}_{I} ( t ) + {F}_{R} ( t ) ) \vert\end{matrix} + \\ \begin{matrix}\vert \frac{1}{{A}_{S}} [ ( 1 + {y}^{2} ) {F}_{I} ( t - b ) + {\sum }_{i = 1}^{n} ( 1 - {y}^{2} ) \\ {( - y )}^{2 t} {F}_{I} ( t - ( 2 i + 1 ) b ) ] - \frac{1}{{A}_{S}} ( {F}_{T} ( t ) \vert\end{matrix}\end{matrix} \} ,$$

where in the formula: n is times of the stress wave undergoes back and forth reflection in the test-piece over time t, n=INT (t/(2b)). The parameter obtained from the above formula at that moment, and y is a dimensionless parameter constructed to make the formula in the preset optimization algorithm hold true. K represents a summation (or iterative) variable/index, which can start from the integer 0 and gradually increase to n, used to traverse each term in the summation expression to obtain the stress distribution function σ_{S}(t,x) of the test-piece at t that moment, as follows:

$${σ}_{S} ( t , x ) = \frac{( y + 1 )}{{A}_{S}} [ {\sum }_{k = 0}^{n} {y}^{2 k} {F}_{I} ( t - \frac{xb}{{L}_{S}} - 2 kb ) - {\sum }_{k = 0}^{n} {y}^{2 k + 1} {F}_{I} ( t + \frac{xb}{{L}_{S}} - 2 ( k + 1 ) b ) ]$$

where in the formula, b represents the time it takes for the stress wave to pass through the test-piece.

The obtained stress distribution function σ_{S}(t,x) is substituted into the reconstruction model mentioned above, the incident wave F₁(t), reflected wave F_{R} (t), and transmitted wave F_{T} (t) data extracted from the incident bar and transmitted bar in SHPB experiments are input, and the stress, strain, and strain rate of the test-piece at the corresponding time are obtain. The stress-strain curve of the test-piece from its stress and strain is reconstructed, and then the mechanical properties such as yield strength, compressive strength, and elastic modulus of the test-piece material are determined.

Through the above method steps, the fitting and identification of the internal stress law of the test-piece can be achieved by constructing and optimizing the behavior through the objective function based on the dynamic response data at the end, thereby obtaining a stress distribution function that can truly reflect the internal stress gradient and overall bearing capacity of the test-piece.

In an implementation mode, in step of substituting the stress distribution function into the dynamic response reconstruction model for calculation and determining the target stress data of the target test-piece, it further includes inputting the stress distribution function as an input parameter into the dynamic response reconstruction model, calculating the stress response of the target test-piece during dynamic loading, and obtaining a force response calculation data; extracting and processing characteristics of an internal force variation of the target test-piece over time and characteristics along a spatial position based on the force response calculation data, and obtaining time distribution feature data and spatial distribution feature data; analyzing an overall stress state of the target test-piece based on the time distribution feature data and the spatial distribution feature data and obtaining the target stress data.

In the embodiments of the present disclosure, the above force response calculation data may be the data results obtained through the model solving process based on the stress distribution mean of the target test-piece stress testing platform, which inputs the stress distribution function into the dynamic response reconstruction model and characterizes the force change law of the target test-piece during the dynamic loading process, including but not limited to the temporal changes of the internal stress of the test-piece, the attenuation trend of stress along the propagation direction, the position of the peak stress, and the overall characteristics of the stress response changing with the loading process.

In an implementation mode, the target test-piece stress testing platform based on the mean stress distribution can perform feature analysis and data screening on the stress variation laws contained in the force response calculation data, and separate the time variation characteristics and spatial position variation characteristics used to describe the internal dynamic mechanical state of the test-piece from the calculation data.

In an implementation mode, the above-mentioned time distribution feature data can refer to the feature information of the variation of the stress on the test-piece over time extracted from the force response calculation data, which is used to describe the evolution trend of the internal stress of the test-piece during the dynamic loading process.

The spatial feature distribution data mentioned above can refer to the feature data extracted from the force response calculation data to describe the spatial position variation law of the internal force of the test-piece. It can also reflect the force magnitude, peak force variation trend, and attenuation behavior along the propagation path of the shock wave at different positions during the propagation process inside the test-piece.

In an implementation mode, it can also identify the differences in load-bearing capacity of the test-piece at different internal spatial points. For example, one side closer to an impact surface may experience higher instantaneous impact forces, and a deeper internal area gradually decreases in load-bearing capacity due to propagation attenuation; or if there is a phenomenon of force concentration in a certain local area, it may become a potential location for damage.

In an implementation mode, the target test-piece stress testing platform based on the mean stress distribution described above comprehensively analyzes and identifies the mechanical laws of the overall stress state of the test-piece during dynamic loading based on time distribution feature data and spatial feature distribution data so as to achieve the goal of analyzing data and obtaining target stress data.

In an implementation mode, the stress concentration area of the test-piece, the overall peak stress state, the dynamic stress evolution trend, and whether there are structural weak points can be identified to generate target stress data for uniformly expressing the overall bearing capacity of the test-piece.

In an implementation mode, in step of analyzing an overall stress state of the target test-piece based on the time distribution feature data and the spatial distribution feature data and obtaining the target stress data, it further includes determining a first force feature data based on the time distribution feature data, and the first force feature data is used to reflect the dynamic stress variation law of the target test-piece; determining a second force feature data based on the spatial distribution feature data, and the second force feature data is used to reflect an internal force distribution state of the target test-piece; obtaining the target stress data based on a correlation calculation between the first force feature data and the second force feature data, and characterizing the overall stress state of the target test-piece using the target stress data.

In the embodiments of the present disclosure, the first force feature data mentioned above may refer to dynamic characteristic parameters determined based on time distribution feature data, which are used to reflect the temporal variation law of the force on the target test-piece during dynamic loading. These parameters can be obtained by calculating the internal force variation behavior corresponding to each time point in the force response calculation results of the target test-piece, including a force growth stage, a peak appearance stage, a peak attenuation process, and a stable stage after loading.

In an implementation mode, the first force feature data can reflect the dynamic force evolution process of the test-piece under impact loading, such as whether the rate of stress increase over time is fast; is there a delay in the occurrence of peak force; is there a clear downward or softening trend after the peak.

By identifying the changes in these time dimensions, the first force feature data can be used to determine the response ability, energy absorption characteristics, and strength variation trend of the test-piece under dynamic stress.

The second force feature data can refer to the characteristic parameters determined based on spatial distribution feature data, which are used to reflect the differences in force states at different spatial positions inside the target test-piece during dynamic loading. By analyzing the force calculation results at multiple positions inside the target test-piece, the internal force distribution pattern of the test-piece can be presented, including stress concentration zone, stress gradient change zone, and stress attenuation zone.

By identifying the peak levels and trends of internal forces at different spatial locations, the second force feature data can be used to analyze the distribution of the internal structural load-bearing capacity of the test-piece, thereby providing a basis for determining whether there are potential failure locations in the test-piece under dynamic stress conditions.

In an implementation mode, the test-piece stress data processing platform based on the mean stress distribution can also perform correlation calculations on two different dimensions of stress feature data, combining the temporal behavior characteristics such as the peak stress occurrence time, stress duration, and stress attenuation rate of the test-piece with the spatial behavior characteristics such as the peak stress size and stress gradient attenuation phenomenon in different internal zones, so that the formation process of bearing capacity of the test-piece has complete structural information.

Through the above method steps, not only can the overall state inference error caused by traditional testing methods due to single point measurement at the end be avoided, but also a unified parameter basis can be provided for the calculation of target stress data. The target stress data obtained under the joint expression of time response and spatial distribution characteristics not only breaks away from the traditional single judgment method based on end face stress measurement for equivalent calculation, but also reveals the internal bearing essence of the test-piece in a more realistic way, rendering the dynamic evaluation results more compatible with the actual working stress environment.

In an implementation mode, in step of testing and judging the applicability or failure risk of the target test-piece under preset working conditions based on the target stress data, it further includes constructing a dynamic stress-strain response curve of the target test-piece based on the target stress data; determining a mechanical performance data of the target test-piece under preset working conditions based on the dynamic stress-strain response curve, and the mechanical performance data includes stress threshold, structural limit, and deformation characteristics; and testing and judging the applicability or failure risk of the target test-piece under preset working conditions based on the stress threshold, structural limit, and deformation characteristics of the target test-piece under preset working conditions.

In the embodiments of the present disclosure, the test-piece stress data processing platform based on the mean stress distribution can further test and judge the applicability or failure risk of the test-piece under preset working conditions after obtaining the target stress data of the target test-piece.

In an implementation mode, based on the target stress data obtained from the dynamic response reconstruction model in this embodiment, the test-piece stress data processing platform based on the mean stress distribution can associate and map the stress response values and deformation behavior of the corresponding test-piece during impact loading, and construct the dynamic stress-strain response curve of the target test-piece.

Through this curve, the load-bearing capacity, deformation energy absorption capacity, and structural toughness behavior of the test-piece under high strain rate conditions can be intuitively reflected, thereby providing a data basis for subsequent structural safety assessments. For example, when the target test-piece exhibits a clear nonlinear deformation path or a decreasing trend in strength peak, its corresponding stress-strain curve can show a significant inflection point or softening segment, indicating the risk of material failure.

In an implementation mode, the test-piece stress data processing platform based on the mean stress distribution can identify key dynamic mechanical performance data of the target test-piece under preset working conditions based on the dynamic stress-strain response curve, including but not limited to:
stress threshold: a critical stress point at which the test-piece yields or undergoes performance changes under dynamic loading conditions;
structural limit: a highest load-bearing capacity or a critical strength position that the corresponding test-piece can withstand under impact;
deformation characteristics: corresponding to the plastic deformation capacity, failure turning behavior, and structural stiffness change trend of the test-piece during the impact process;
where the stress threshold can be identified by the inflection point or yield point of the curve, the structural limit can reflect the peak position of the curve, and the deformation characteristics can be determined based on the changes in the nonlinear or softening section of the curve. The above mechanical performance data is used to quantify the true load-bearing capacity and deformation energy absorption performance of test-piece under impact conditions, and can serve as important indicators for applicability testing and risk screening.

In this embodiment, the stress data processing platform based on the mean stress distribution can also judge whether the target test-piece meets the bearing requirements or has a risk of performance degradation under preset working conditions based on performance data such as stress threshold, structural limit, and deformation characteristics.

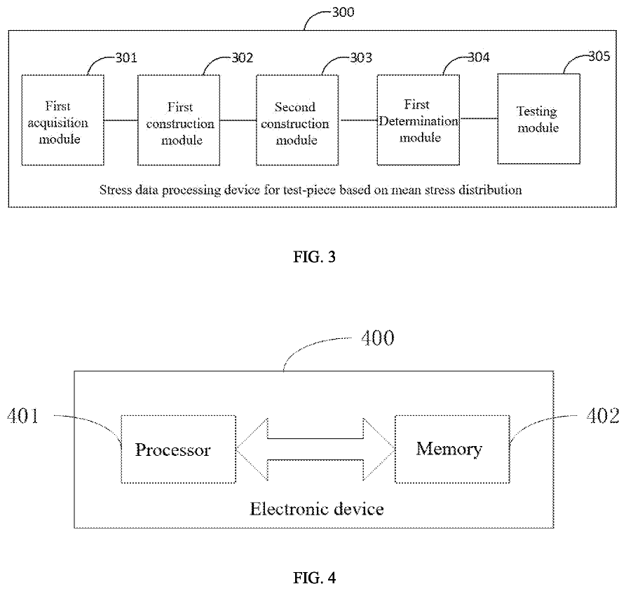

In an implementation mode, a stress data processing device for test-piece based on mean stress distribution 300 is provided, which corresponds one-to-one with the stress data processing method for test-piece based on the mean stress distribution. As shown in FIG. 3, a first acquisition module 301 of the stress data processing device for test-piece based on mean stress distribution is configured to determine a mean stress distribution of a target test-piece; a first construction module 302 is configured to construct a dynamic response reconstruction model based on the mean stress distribution; a second construction module 303 is configured to construct a stress distribution function of the target test-piece through a preset optimization algorithm; a first determination module 304 is configured to substitute the stress distribution function into the dynamic response reconstruction model for calculation, and determine a target stress data of the target test-piece; and a testing module 305 is configured to test and judge applicability or failure risk of the target test-piece under preset working conditions based on the target stress data.

The specific limitations of the stress data processing device for test-piece based on mean stress distribution can be found in the previous section on the limitations of the stress data processing method for test-piece based on the mean stress distribution, which will not be repeated here. The various modules in the stress data processing device for test-piece based on mean stress distribution can be fully or partially implemented through software, hardware, and their combinations. The above modules can be embedded in hardware form or independent of a processor in a computer device, or stored in software form in a memory of the computer device, so that the processor can call and execute the corresponding operations of the above modules.

In the embodiment of the present application, as shown in FIG. 4, an electronic device 400 includes a memory 402, a processor 401, and a computer-readable instruction that is stored in the memory 402 and executable on the processor 401. When the processor 401 executes the computer-readable instruction, it implements steps of the stress data processing method for test-piece based on the mean stress distribution.

In the embodiment of the present application, a readable storage medium is provided, which stores computer-readable instruction. When the computer-readable instruction is executed by a processor, steps of the stress data processing method for test-piece based on the mean stress distribution are implemented.

Those skilled in the art can understand that the implementation of all or part of the processes in the above embodiments can be completed by instructing relevant hardware through computer programs, and can be stored in the computer-readable storage medium. When executed, the program may include the processes of the embodiments of the above methods. The storage medium can be magnetic disk, optical disk, Read Only Memory (ROM) or Random Access Memory (RAM), etc.

The above disclosure is only preferred embodiment of the present disclosure and cannot be used to limit the scope of the present disclosure. Therefore, equivalent changes made according to the claims of the present disclosure still fall within the scope of the present disclosure.

What is claimed is:

1. A stress data processing method for test-piece based on the mean stress distribution, comprising:
determining a mean stress distribution of a target test-piece, by a stress data processing platform based on the mean stress distribution;
constructing a dynamic response reconstruction model based on the mean stress distribution, by the stress data processing platform based on the mean stress distribution;
constructing a stress distribution function of the target test-piece through a preset optimization algorithm, by the stress data processing platform based on the mean stress distribution;
substituting the stress distribution function into the dynamic response reconstruction model for calculation, and determining a target stress data of the target test-piece, by the stress data processing platform based on the mean stress distribution; and
testing and judging applicability or failure risk of the target test-piece under preset working conditions based on the target stress data, by the stress data processing platform based on the mean stress distribution;
wherein determining a mean stress distribution of a target test-piece comprises:
real time collecting a force response of the target test-piece at a preset position, obtaining a dynamic response data of the target test-piece, and the dynamic response data is configured to describe an end force change of the target test-piece; and
calculating and processing an internal stress distribution of the target test-piece based on the dynamic response data to obtain the mean stress distribution;
wherein the dynamic response reconstruction model comprises a stress reconstruction model, a strain reconstruction model, and a strain rate reconstruction model;
wherein constructing a dynamic response reconstruction model based on the mean stress distribution comprises:
determining target correlation parameters of the target test-piece based on the mean stress distribution, and the target correlation parameters are configured to uniformly characterize a dynamic stress state of the target test-piece; and
constructing the stress reconstruction model, the strain reconstruction model, and the strain rate reconstruction model respectively based on the target correlation parameters;
wherein constructing a stress distribution function of the target test-piece through a preset optimization algorithm comprises:
determining an objective function based on the dynamic response data and stress distribution mean of the target test-piece, and the objective function is configured to characterize the internal stress distribution characteristics of the target test-piece; and
solving and calculating the objective function using the preset optimization algorithm to obtain the stress distribution function.

2. The stress data processing method for test-piece based on the mean stress distribution according to claim 1, wherein substituting the stress distribution function into the dynamic response reconstruction model for calculation and determining a target stress data of the target test-piece comprises:
inputting the stress distribution function as an input parameter into the dynamic response reconstruction model, calculating the stress response of the target test-piece during dynamic loading, and obtaining a force response calculation data;
extracting and processing characteristics of an internal force variation of the target test-piece over time and characteristics along a spatial position based on the force response calculation data, and obtaining time distribution feature data and spatial distribution feature data; and
analyzing an overall stress state of the target test-piece based on the time distribution feature data and the spatial distribution feature data and obtaining the target stress data.

3. The method for processing stress data of a test-piece based on the mean stress distribution according to claim 2, wherein analyzing an overall stress state of the target test-piece and obtaining the target stress data based on the time distribution feature data and the spatial distribution feature data comprises:
determining a first force feature data based on the time distribution feature data, and the first force feature data is configured to reflect the dynamic stress variation law of the target test-piece;
determining a second force feature data based on the spatial distribution feature data, and the second force feature data is configured to reflect an internal force distribution state of the target test-piece; and
obtaining the target stress data based on a correlation calculation between the first force feature data and the second force feature data and characterizing the overall stress state of the target test-piece using the target stress data.

4. The method for processing stress data of a test-piece based on the mean stress distribution according to claim 1, wherein testing and judging applicability or failure risk of the target test-piece under preset working conditions based on the target stress data comprises:
constructing a dynamic stress-strain response curve of the target test-piece based on the target stress data;
determining a mechanical performance data of the target test-piece under preset working conditions based on the dynamic stress-strain response curve, and the mechanical performance data comprises stress threshold, structural limit, and deformation characteristics; and
testing and judging the applicability or failure risk of the target test-piece under preset working conditions based on the stress threshold, structural limit, and deformation characteristics of the target test-piece under preset working conditions.

5. A stress data processing device for test-piece based on mean stress distribution, wherein it is configured to implement the stress data processing method for test-piece based on the mean stress distribution according to claim 1, and the stress data processing device comprises:
a first acquisition module, configured to determine the mean stress distribution of the target test-piece;
a first construction module, configured to construct the dynamic response reconstruction model based on the mean stress distribution;
a second construction module, configured to construct the stress distribution function of the target test-piece through the preset optimization algorithm;
a first determination module, configured to substitute the stress distribution function into the dynamic response reconstruction model for calculation, and determine the target stress data of the target test-piece; and
a testing module, configured to test and judge applicability or failure risk of the target test-piece under preset working conditions based on the target stress data.

6. An electronic device, which comprises a memory, a processor, and a computer program that is stored on the memory and executable on the processor, wherein the processor executes steps of the stress data processing method for test-piece based on the mean stress distribution according to claim 1 when executing the computer program.

7. A computer-readable storage medium, wherein a computer program is stored on the computer-readable storage medium, and the computer program, when executed by a processor, implements steps of the stress data processing method for test-piece based on the mean stress distribution according to claim 1.
